(19) United States
(12) Patent Application Publication
(10) Pub. No.: US 2015/0164404 A1
(43) Pub. Date: Jun. 18, 2015

(54) SYSTEM AND METHOD FOR DETECTING PREECLAMPSIA

(71) Applicant: Convergent Engineering, Inc., Newberry, FL (US)

(72) Inventors: Tammy Y. Euliano, Gainesville, FL (US); Neil Russell Euliano, II, Gainesville, FL (US); Shalom Darmanjian, Newberry, FL (US)

(21) Appl. No.: 14/401,657

(22) PCT Filed: May 24, 2013
(86) PCT No.: PCT/IB2013/001729

§ 371 Date: Nov. 17, 2014

Related U.S. Application Data
(60) Provisional application No. 61/650,616, filed on May 23, 2012.

(51) Int. Cl. A61B 5/00; A61B 5/024; A61B 5/021; A61B 5/0444; A61B 5/1455

(52) U.S. Cl. CPC A61B 5/4356; A61B 5/0444; A61B 5/14551; A61B 5/02125; A61B 5/02416; A61B 5/02405; A61B 5/02411; A61B 5/6824; A61B 5/7275; A61B 5/7278

(57) ABSTRACT

A system and method for detecting preeclampsia in a patient is provided. Also provided is a system and method for diagnosing preeclampsia in a patient prior to the detection of conventional symptoms and/or clinical signs associated with preeclampsia. The preeclampsia detection system of the invention comprises at least one sensor and a processor comprising a preeclampsia recognizer. In certain embodiments, the system farther comprises a user interface.

### CROSS-REFERENCE TO A RELATED APPLICATION

This application claims the benefit of U.S. provisional application Ser. No. 61/650,616, filed May 23, 2012, which is incorporated herein by reference in its entirety.

### BACKGROUND OF INVENTION

Preeclampsia is a major cause of maternal and neonatal morbidity and mortality around the world, responsible for approximately 76,000 maternal and 500,000 infant deaths per year (Preeclampsia Foundation, “About Preeclampsia,” (2012)). Its heterogeneous presentation complicates diagnosis and institution of therapy, while causing unnecessary treatment in many others. Left untreated, preeclampsia can rapidly and unexpectedly worsen to life-threatening hypertension, seizures, pulmonary edema and coagulation system effects. Early recognition of the symptoms, treatment of hypertension, prevention of seizures with magnesium and progression to delivery (the only cure, even if preterm) minimizes mortality. Recent studies of angiogenic factors as diagnostic tests hold promise, but at substantial cost. Currently there are no readily available, non-invasive tests to diagnose preeclampsia.

Preeclampsia affects 5-8% of pregnancies in the US, with its complications accounting for 18% of maternal deaths. Maternal and fetal morbidity present an additional, if immeasurable cost. The pathophysiology of preeclampsia remains an area of intense research, the outcome of which should lead to novel prevention and treatment strategies. In the meantime there are methods to reduce morbidity and mortality such as blood pressure control, magnesium sulfate to prevent eclamptic seizures and delivery of the premature infant in a center with necessary capabilities. Diagnosis of preeclampsia in the previously normotensive patient presenting with typical symptoms (new-onset hypertension and proteinuria) is uncomplicated. However, nearly one-third of preeclamptics do not present so clearly (von D P et al. “Prediction of adverse maternal outcomes in preeclampsia: development and validation of the full PIERS model.” Lancet Jan. 15, 2011; 377(9761):219-27). In fact even in those with seizures (eclampsia), almost half (43%) were not previously diagnosed with both hypertension and proteinuria (Douglas K A, Redman C W. Eclampsia in the United Kingdom. BMJ Nov. 26, 1994; 309(6966):1395-400. PMCID:PMC2541348). Development of a low-cost, portable, reliable device to diagnose preeclampsia would reduce complications and mortality.

While many groups have investigated various ways to predict or detect preeclampsia, the vast majority of techniques require expensive equipment or laboratory tests. Of recent interest is angiogenic markers, primarily placental growth factor and soluble Fms-like tyrosine kinase-1 (Benton S J, et al. “Angiogenic factors as diagnostic tests for preeclampsia: a performance comparison between two commercial immunoassays.” Am. J Obstet. Gynecol. November 2011; 205(5):469-8). Unfortunately, cost and assay availability are primary limitations to ensuring diagnosis of preeclampsia via detection and/or quantification of such markers. Identification of the cardiovascular changes unique to preeclampsia may provide an alternative for diagnosis.

Maternal arterial characteristics in preeclampsia have been evaluated using non-invasive applanation tonometry in which a device, applied to the radial artery, extracts the pressure waveform; analysis of the reflecting waves infers vascular resistance. This device is expensive, requires training, and suffers from reproducibility issues, but the studies provide useful insight into the physiology. In a cross-sectional study of 69 normotensive and 54 preeclamptic pregnant women, Kaihura et al. detected a 20% difference in the carotid to femoral median pulse wave velocity; and a 10% difference between carotid and radial (Kaihura C et al. “Maternal arterial stiffness in pregnancies affected by preeclampsia.” Am. J Physiol Heart Circ. Physiol August 2009; 297(2):H759-H764). The group deduced an increase in maternal arterial stiffness with preeclampsia.

Similarly Arioz et al. studied 60 consecutive pregnant women in the third trimester of pregnancy with digital photoplethysmography and 24-hour ambulatory blood pressure (Arioz D T et al. “Arterial stiffness and dipper/nondipper blood pressure status in women with preeclampsia.” Adv. Ther. September 2008; 25(9):925-34). Thirty women were preeclamptic by standard criteria, a surprisingly high incidence. For this study, the group calculated the arterial stiffness index (SI) from the digital volume pulse (DVP) obtained with pulse oximetry. This study monitored changes in SI in preeclamptic patients. Unfortunately, this study failed to offer any suggestions for determining those patients likely to develop preeclampsia or those patients with non-symptomatic preeclampsia. Moreover, changes in SI alone do not necessarily provide an accurate means for determining those patients likely to develop preeciampsia or diagnosing those patients with non-symptomatic preeclampsia.

Described in 2000 by Millasseau et al., the first derivative with respect to time of the DVP is used to identify the inflection point (similar to the dichrotic notch in an arterial waveform) (Millasseau S C et al. “Contour analysis of the photoplethysmographic pulse measured at the finger.” J. Hypertens. August 2006; 24(8):1449-56). The time between the systolic peak and this notch is calculated and used to derive the SI as body height/ΔT. Arioz et al. (Ibid.) identified a 50% increase in SI (5.9±0.8 m/s vs. 8.8±1.2) with preeclampsia. Most recently, Avni et al. examined 100 pregnant patients including preeclamptic, chronic hypertensive, and normotensive parturients. Their findings agree with those above, identifying an increase in aortic stiffness, as assessed by pulse wave analysis with applanation tonometry (Avni B et al. “Aortic stiffness in normal and hypertensive pregnancy.” Blood Press February 2010; 19(1):11-5). These studies used devices impractical for routine use in clinics, especially by less trained personnel. Noninvasive applanation tonometry is performed in a device applied to the radial artery that extracts the pressure waveform. This device is expensive, requires training, and suffers from reproducibility issues, but the studies provide useful insight into the physiology.

In another example, a method for monitoring preeclampsia involves analysis of cardiovascular oscillations noninvasively via a finger cuff (H Malberg et al., “Analysis of cardiovascular oscillations: A new approach to the early prediction of pre-eclampsia,” Chaos 17, 015113 (2007)). According to the Malberg et al. system, the finger cuff continuously monitors blood pressure and extracts time series of beat-to-beat intervals, and systolic and diastolic blood pressures (Portapres device, BMI-TNO). The Malberg et al. system is rather complex and illustrated in FIG. 1. Malberg et al. observed 96 patients with abnormal uterine perfusion identified by doppler sonography, 24 of whom eventually developed preeclampsia. They utilized a variety of entropy measures and statistical methods to analyze heart rate (HR) and blood pressure variability, etc.

Another method (Khalil A. et al. (2009) “Pulse Wave Analysis in Normal Pregnancy: A Prospective Longitudinal Study.” PLoS ONE 4(7): e6134. doi:10.1371/journal.pone.0006134) involves pulse wave analysis. Pulse wave analysis provides valuable information in hypertension and vascular disease. Khalil et al. used a tonometer to measure arterial pulse waves and, following pulse wave analysis, evaluated changes in pulse wave analysis parameters to investigate whether these parameters are affected by ethnicity. Unfortunately, tonometers are expensive and difficult to use, with reliability and repeatability issues.

Khalil A. et al. (“Pulse wave analysis: a preliminary study of a novel technique for the prediction of pre-eclampsia.” BJOG 2009; 116:268-277) also investigated whether first-trimester arterial pulse wave analysis can predict preeclampsia. In this study, 11-14 weeks of gestation pulse waves were measured with tonography. Arterial PWA was performed as follows: the radial artery was gently compressed with the tip of the tonometer at the site of maximal pulsation. This tonometer contains a micromanometer that provides a very accurate recording of the pressure within the radial artery (Millar Instruments, Houston, Tex., USA). Unfortunately, as indicated above, tonometers are expensive and difficult to use, with reliability and repeatability issues.

In a further study, radial artery applanation tonometry was utilized (Spasojevic et. al. “Peripheral arterial pulse wave analysis in women with pre-eclampsia and gestational hypertension,” BJOG: an International Journal of Obstetrics and Gynaecology, November 2005, Vol. 112, pp. 1475-1478). Women in the third trimester of pregnancy with newly developed preeclampsia (PE) (n=27) or gestational hypertension (GH) (n=33) were studied by radial artery applanation tonometry. Spasojevic et al. determined hypertension was of equal severity in PE and GH and concluded measurement of Augmentation Index (AI) gives clear separation of established PE both from normal pregnancy and from uncomplicated GH. As indicated above, tonometers are, unfortunately, expensive and difficult to use, with reliability and repeatability issues.

### BRIEF SUMMARY OF THE INVENTION

The subject invention provides a non-expensive, non-invasive system and method for predicting and/or determining preeclampsia in a patient. While the disease can begin benignly enough with a headache, life-threatening hypertension, seizures, pulmonary edema and coagulation system effects can occur rapidly and unexpectedly. Even in developed countries, complications and deaths occur as a result of preeclampsia. Therefore, early recognition of the symptoms, treatment of hypertension, prevention of seizures and progression to delivery (the only cure, even if preterm) minimizes mortality. Unfortunately many low-income countries lack access to the proper test (blood pressure and urine protein testing) to even diagnose preeclampsia once it manifests, let alone predict it. In addition to operating as an early-warning prediction system, the subject invention detects preeclampsia after onset (and, in certain instances, prior to detection of conventional symptoms associated with preeclampsia), facilitating treatment and/or delivery or transfer planning.

A sensor device is disclosed that includes sensors adapted to be worn on a patient's body. The sensors include those that generate information indicative of detected physiological parameters of the patient. In one embodiment, a sensor device is provided comprising a pulse oximeter probe and at least one ECG sensor, wherein the sensors generate data indicative of photoplethysmographic (PPG) measurements and electrocardiogram (ECG) signal(s), respectively. The sensor device can be produced from inexpensive and/or reusable sensor technologies. In certain embodiments, the sensor device is portable and/or wearable.

The sensor device can further include a housing adapted to be worn on a patient's body, wherein the housing supports the sensors or wherein at least one of the sensors is separately located from the housing. The sensor device may further include a flexible body supporting the housing having first and second members that are adapted to wrap around a portion of the patient's body. The flexible body may support one or more of the sensors. The sensor device may further include wrapping means coupled to the housing for maintaining contact between the housing and the patient's body, and the wrapping means may support one or more sensors.

The sensor device can include any one or more of the following: a processor that receives at least a portion of data generated by the sensors and is adapted to generate derived data related to the detection and/or prediction of preeclampsia; a display for communicating information regarding the data collected by the sensor device; a user interface. In a preferred embodiment, as illustrated in FIG. 2, the sensor device is a portable or wearable device provided on a wrist strap.

The invention is also directed to a system for predicting and/or diagnosing preeclampsia in a patient. The system of the invention comprises a sensor device, a processor adapted to generate derived data from the information provided by the sensor device, and a user interface for reporting the likelihood of current or future preeclampsia. The sensor device can include the processor or the processor may alternatively be external to the sensor device. The reports from the user interface can be provided to the patient and/or to clinical personnel. The system can be customized based on local clinical infrastructure and cultural differences and can be programmed to advise on follow-up and/or therapy, including reprogramming as recommendations change. Furthermore, data collection to better understand the effectiveness of various treatments is also feasible. The system could also transmit data to a central server which performs the required processing to interpret the data using the latest algorithms. The results of the processing along with location- or cultural-specific therapy recommendations could then be transmitted back to the device, the user's cell phone, or other communication device.

Advantages of the invention include one or more of the following. The system allows patients and/or clinicians to conduct a low-cost, comprehensive, real-time monitoring for preeclampsia. Use of the subject invention can result in diagnosis and treatment of preeclampsia and, in some cases, predict preeclampsia before symptoms are detected. Because the system is non-invasive and, in certain embodiments, has no disposable parts, its cost per patient is very small, perhaps a penny per patient test or less.

The subject invention is simple to use and modular. For example, the sensor device can be built in many easy to use form factors including an armband that simply straps around the wrist of a patient. After a few minutes of data collection, a display will indicate the likelihood of present or future onset of preeclampsia. Additionally, the information can be sent via multiple methods to a computer, website, external database, or other location for analysis, storage, and/or further processing. Untrained or minimally trained clinical personnel (or the patient) can use the system.

The system provides real time and point of care prediction and/or detection of preeclampsia. There is no required lab work or any delay in test result reporting. The system is placed on the patient and within a few minutes provides the results of the test.

In particular, the system is easy to maintain. There is no calibration, chemical testing, or other complicated methods necessary. Only recharging of the battery or application of power is required for the sensor device.

The system of the invention preferably comprises a portable and/or wearable sensor device. The sensor device may be small and easily worn by the patient and can non-invasively capture data on plethysmographic waveform and ECG to report detection and/or prediction of preeclampsia. Preferably, the sensor device is a cuff that can be worn on the arm or the wrist.

In a preferred embodiment, the system comprises a sensor device that captures data on plethysmographic waveform and single-channel ECG to non-invasively detect preeclampsia, as well as to differentiate between mild and severe preeclampsia. The subject system may be used in labor & delivery suites and emergency departments for early diagnosis of preeclampsia and initiation of magnesium therapy where indicated.

The subject system facilitates the diagnosis of preeclampsia, distinguishing it from other forms of hypertension that may present in labor and delivery. This enables magnesium therapy to be initiated appropriately, in only those patients who will benefit. The system also identifies parturients at prenatal visits who are at high risk of developing preeclampsia, and distinguishes those who will develop the more severe form. Such a device enhances patient care by:
allowing transfer of such patients to an appropriate-level provider (e.g. home delivery becomes less desirable).
encouraging directed education of the identified high-risk patient regarding warning signs and increased frequency of blood pressure monitoring.
enabling healthcare providers to plan for more frequent evaluations of the fetus and the potential for a preterm delivery. For example, if severe complications are predicted, (a) more frequent prenatal visits and observation of fetal growth may be indicated, (b) antenatal steroids for lung maturation may be considered, and (c) development of contingency plans for delivery at a center with a neonatal intensive care unit (NICU) and availability of blood products should HELLP syndrome (a clotting disorder) develop.
facilitating research protocols into prevention and treatment strategies that are best implemented in a population of known risk, e.g. administration of dietary supplements. This could be investigated at reasonable cost in the subgroup of patients identified with this technology.

With early prediction capabilities, the subject invention can be part of routine screening in medical clinics that offer prenatal care. Once preeclampsia is identified, the system could improve outcomes for both mother and fetus by enabling (1) directed patient education, (2) increased prenatal monitoring, (3) administration of supplements that may reduce preeclampsia severity, and (4) delivery planning, including transportation to an appropriate facility. Furthermore, the system may include real-time updates on recommendations from American Congress of Obstetricians and Gynecologists (ACOG), and could suggest possible study protocols. The system also has a large potential for use in research of preeclampsia and treatments. For example, use of the system as an accurate screening device in clinical trials assessing treatments for preeclampsia could provide significant cost and resource savings.

### BRIEF DESCRIPTION OF DRAWINGS

FIG. 1 illustrates one embodiment of the prior art.

FIG. 2 illustrates an embodiment of the invention wherein an interface cable of the invention is operatively connected to a laptop PC or other communication device to transmit or process data.

FIG. 3 illustrates a radial basis function network for preeclampsia detection.

FIG. 4 illustrates a typical ECG and PPG waveform with features of each waveform and timing parameters.

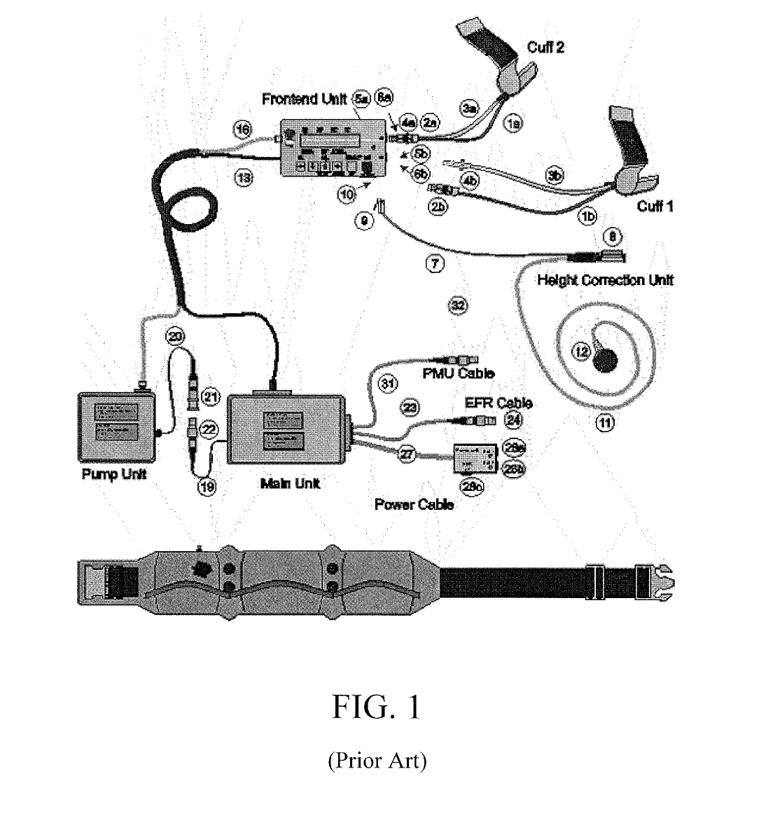

### DETAILED DISCLOSURE

A system and method for detecting preeclampsia in a patient is provided. Also provided is a system and method for diagnosing preeclampsia in a patient prior to the detection of conventional symptoms or clinical signs associated with preeclampsia. Conventional symptoms associated with preeclampsia include, but are not limited to, swelling, abdominal pain, seizures, sudden weight gain, headaches and changes in vision. Typical clinical signs include hypertension, protein in the urine, and hyperreflexia. The preeclampsia detection system of the invention comprises a sensor device and a processor comprising a preeclampsia recognizer. In certain embodiments, the system further comprises a user interface.

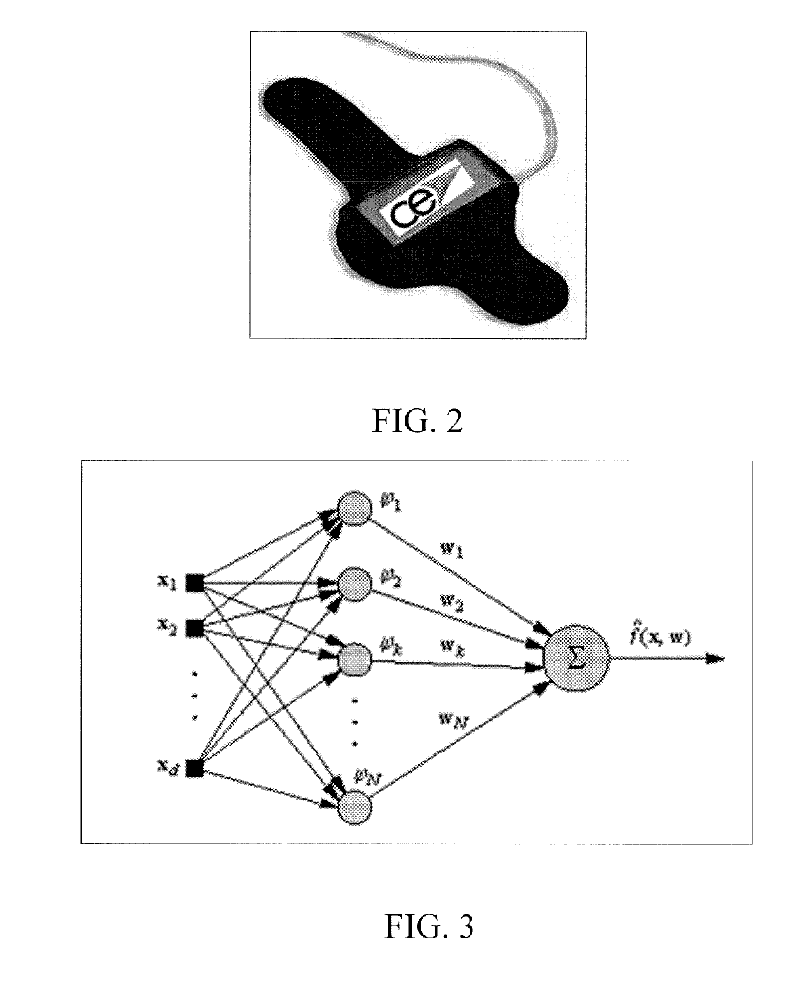

FIG. 2 shows an exemplary sensor device. The sensor device can operate in a home, clinic or hospital. In certain embodiments, the sensor device comprises one or more sensors situated together as a single unit to be non-invasively worn by or applied to a patient. In a related embodiment, the one or more sensors are situated within a single housing unit or device. A preferred embodiment of the sensor device comprises a simple wrist/arm band that is held in place via elastic band or Velcro strap, wherein situated on the band are one or more sensors. Because the intelligent algorithms of the system of the invention require only a single photoplethysmography (PPG) channel and a single electrocardiogram (ECG) channel, the sensors can comprise optical transducer(s) and electrode sensor(s). Preferably, two or more electrodes and one or more optical transducers are used.

An optical transducer can be a sensor comprising a light source and a photo-detector. The light source can be light-emitting diodes (LED) that generate red (λ=about 630 nm) and/or infrared (λ=about 900 nm) radiation, for example. The light source and the photo-detector are slidably adjustable and can be moved along the wrist/arm band to optimize beam transmission and pick up. As the heart pumps blood through the patient's finger, blood cells absorb and transmit varying amounts of the red and infrared radiation depending on how much oxygen binds to the cells' hemoglobin. The photo-detector detects transmission at the predetermined wavelengths, for example, red and infrared wavelengths, and provides the detected transmission to a pulse-oximetry circuit, which may also be located on the wrist/arm band. The output of the pulse-oximetry circuit is digitized into a time-dependent optical waveform (plethysmographic waveform), which is then sent back to the pulse-oximetry circuit for further analysis (e.g., by the processor) and/or further transmission (e.g., to the display). Although standard pulse-oximetry uses two frequencies of light to determine the amount of oxygenated hemoglobin, only one frequency of light is required to create a waveform of blood flow (plethysmography).

The sensor device can include at least one electrode sensor that enables differential ECG to be measured. Contemplated electrode sensors include, but are not limited to, disposable sensors (including sensors that are without gel or pregelled), reusable disc electrodes (including gold, silver, stainless steel, or tin electrodes), headbands, saline-based electrodes, impedance, radio frequency (RF), and acoustic sensors. Contemplated sensors include those used for monitoring electrocardiography (ECG/EKG); electroencephalography (EEG); electromyography (EMG); electronystagmography (ENG); electro-oculography (EOG), printed circuit sensors, electroretinography (ERG), bioimpedance sensors (RF or otherwise) and stethoscope sensors.

The electrical signal derived from an electrode is typically 1 mV peak-peak. In certain embodiments, an ECG amplifier (e.g., a one-channel ECG amplifier or differential amplifier) is provided to amplify the electrical signal by about 100 to about 1,000 times as necessary to render this signal usable for detection.

The sensors of the sensor device can be removable. Further, the sensors can be passive (such as a reader) and store information. Alternatively, or in addition, the sensors can transmit information (e.g., to a processor for analysis purposes).

The sensor electronics and power source of a sensor device are preferably small. The power source can be any portable power source capable of fitting on the sensor device. According to some embodiments, the power source is a portable rechargeable lithium-polymer or zinc-air battery. Additionally, portable energy-harvesting power sources can be integrated into the sensor device and can serve as a primary or secondary power source. For example, a solar cell module can be integrated into the sensor device for collecting and storing solar energy. Additionally, piezoelectric devices or microelectromechanical systems (MEMS) can be used to collect and store energy from body movements, electromagnetic energy, and other forms of energy in the environment or from the patient. A thermoelectric or thermovoltaic device can be used to supply some degree of power from thermal energy or temperature gradients. In some embodiments, a cranking or winding mechanism can be used to store mechanical energy for electrical conversion or to convert mechanical energy into electrical energy that can be used immediately or stored for later.

In a preferred embodiment, the sensor device comprises at least one optical transducer, a pulse-oximetry circuit, at least one electrode, and a one-channel ECG amplifier that is provided in an electronic sensor assembly. The electronic sensor assembly is preferably small in size (approximately 2″×3″) and can be powered by two watch batteries or similar rechargeable technology. As such, this system is very small and can be wearable or portable.

In a related embodiment, the sensor device is a simple armband that contains two metal electrodes (similar to exercise watches or equipment) and one or more optical transducers. More than one optical transducer (photodetector and LED) may be provided on the armband, particularly those optical transducers that are very small and inexpensive, to ensure robust data collection across different band locations and arm sizes.

Alternatively, the system of the invention may comprise more than one sensor device. For example, the preeclampsia detection system can include a sensor device comprising one or more electrodes and another sensor device comprising one or more

PPG sensors. In one embodiment, the system comprises a standard finger pulse oximeter and simple ECG sensor placed anywhere on the body. In a related embodiment, multiple ECG sensors are provided on the maternal abdomen. Information from the electrodes on the maternal abdomen can be used not only to detect and/or predict preeclampsia but also for antepartum and/or intrapartum maternal fetal monitoring as described in U.S. Pat, No. 7,333,850, which is incorporated herein by reference in its entirety. Alternatively, the preeclampsia detection system may include the electrode ECG sensors and interface cable as described in U.S. Pat. No. 7,828,753, which is incorporated herein by reference in its entirety.

A signal conditioning front-end of the preeclampsia detection system amplifies the low level ECG bioelectric signals coming from the electrodes and provides low-impedance signals to a data acquisition module, which can be connected to or be a part of a processor. Active common mode noise suppression is used to reduce or eliminate 60 Hz electric power line noise typically present in signals from human body surface electrodes. The data acquisition module is designed with a low-power and low-noise 24-bit analog-to-digital converter (ADC). This 24-bit ADC provides a very large dynamic range that eliminates input saturation with high level muscle contraction signals, and has very high signal resolution, passing an accurate low-noise signal to the system processor (initially on the smartphone/PC, eventually an embedded processor in the armband). The system processor is used to process the ECG and PPG data streams acquired by the ADCs.

The sensor device preferably implements continuous ECG recording and collection of pulse oximetry waveforms (photoplethysmography, PPG) from various locations on a patient's body. Those locations include, but are not limited to, the finger, wrist, ear, nose, cheek, forehead, chest, abdomen etc. of the patient. For example, an array of sensors may be provided for the abdomen, where the array has a low spatial resolution.

In certain embodiments, the system comprises a user interface. The user interface can be a personal or tablet computer, a cell phone monitor, a PDA monitor, a television, a projection monitor, a visual monitor on the sensor device, or any method of visual display. The preferred user interface in the system is a low power liquid crystal display (LCD) or similar display on the armband.

Signal data from the sensor device(s) (e.g., PPG and ECG signals) are transmitted to a processor. The data can be transmitted periodically or at a later time. This delayed transmission may, without restriction, be utilized to improve battery life by transmitting data transiently, instead of continuously; or to allow for patient monitoring during disconnection from the sensor device.

The processor of the preeclampsia detection system is a device that performs any one or more of the following functions: (1) it stores the signals to memory, such as a flash or SRAM, for subsequent analysis; (2) it stores a number of signals to memory and subsequently transmits them, wired or wirelessly, to a remote computer for preeclampsia detection as described herein and/or display, such as display in real time; or (3) it processes the signals using a software module as described herein to detect preeclampsia in a patient. A variety of microprocessors or other processors may be used herein.

According to one embodiment, a wireless signal transmitter may be utilized between the sensor device(s) and the processor. The wireless signal transmitter can include a data storage device (such as a magnetic hard drive, flash memory card, and the like). Preferably, the wireless signal transmitter includes communications protocols for data representation, signaling, authentication, and error detection that required to send information over a wireless communications channel (i.e., a specific radio frequency or band of frequencies such as Wi-Fi, which consists of unlicensed channels 1-13 from 2412 MHz to 2484 MHz in 5 MHz steps). The wireless signal transmitter is preferably located on or near the sensor device(s). For example, the wireless signal transmitter can be attached to a housing on an armband of the sensor device. Many wireless transmission communications protocols exist and are applicable to the wireless signal transmitter/receiver of this invention, including Bluetooth, Wi-Fi, Zigbie, wireless USB, etc. The wireless transmission of information from the wireless signal transmitter to the wireless signal receiver could be in digital format or in analog format.

In certain embodiments, the wireless signal transmitter (and/or wireless signal receiver) includes an internal power source (i.e., batteries, and the like). Alternatively, the wireless signal transmitter (and/or wireless signal receiver) does not require an internal power source. This can be accomplished with a variety of energy harvesting or wireless power transmission methods such as harvesting of heat, movement, electrical signals from the environment, or inductive coupling. In one embodiment, this is accomplished by using an antenna to convert radiated or inducted power into usable energy for the transmission of the desired signals. For example, the wireless signal transmitter can be an antenna that is commonly used in radio frequency identification tags (or RFID tags), where minute electrical current induced in the antenna by an incoming radio frequency signal provides just enough power for an integrated circuit (IC) in the RFID tag to power up and transmit a response (for example, to a wireless signal receiver of the invention).

In a preferred embodiment, the processor executes one or more software modules to analyze signals from the sensor device. More preferably, the processor is configured to run a preeclampsia recognizer that is used to analyze PPG and ECG signals. For example, PPG and ECG signals can be used as input to a preeclampsia recognizer. A preeclampsia recognizer can comprise one or more classification or prediction models (for the detection and/or prediction of preeclampsia). Such classifiers include, but are not limited to, simple clustering analysis and logistic regression models. Nonlinear models are also envisioned due to their classification and prediction performance, including but not limited to:
Support Vector Machines. Similar to Radial Basis Function Network, this type of model separates the classes with high-dimensional hyper plane using the samples nearest the decision surface to maximize the margin.
Neural Network. Although traditionally a black box modeling tool, neural networks afford an increase in the degrees of freedom to model the aforementioned data non-linearly.
Information theoretic methods. Using these may help in modeling features that are non-Gaussian.
State-spaced methods. These models can identify hidden state information present in the data. Exploiting the temporal-state information may increase performance beyond our static classifier. The Kalman filter (continuous state-space) and Hidden Markov Model (HMM) are two such models that will be implemented.

In one embodiment, the preeclampsia recognizer is a statistical analyzer such as a neural network that has been trained to flag preeclampsia. The neural network can be a back-propagation neural network, for example. In this embodiment, the statistical analyzer is trained with training data where certain signals are determined to be undersirable for the patient. For example, the patient's desirable pattern of PPG and ECG signals or features should be within a well-established range, and any values outside of this range are flagged by the preeclampsia recognizer as a preeclampsia condition. Once the preeclampsia recognizer is trained, the data received by the processor can be appropriately scaled and processed.

In certain embodiments, the preeclampsia recognizer is trained from patient data to optimally separate a variety of patient scenarios, including: preeclamptics from non preeclamptics, mild versus severe preeclamptics, differentiation of preeclamptics from other forms of hypertension such as gestational hypertension, patients likely to eventually have preeclampsia symptoms. In a related embodiment, the preeclampsia recognizer is a Radial Basis Function Network (RBF, see FIG. 3) with a linear output to discriminate/detect preeclamptics versus controls.

In certain related embodiments, the patient data feature set consists of parameters from four different physiologic classes: A) heart rate, B) pulse transit time (PTT, correlates with blood pressure), C) augmentation indices, and D) oximetry. Multiple parameters from each class capture different representations of the fundamental data (e.g., heart rate or PTT variability), and combinations of parameters are also derived (e.g., change in PTT per change in heart rate). Using the different covariates, a high-dimensional feature vector is assembled as input into the preeclampsia recognizer (e.g., RBF classifier). Any combination of these parameters may provide useful information to the system.

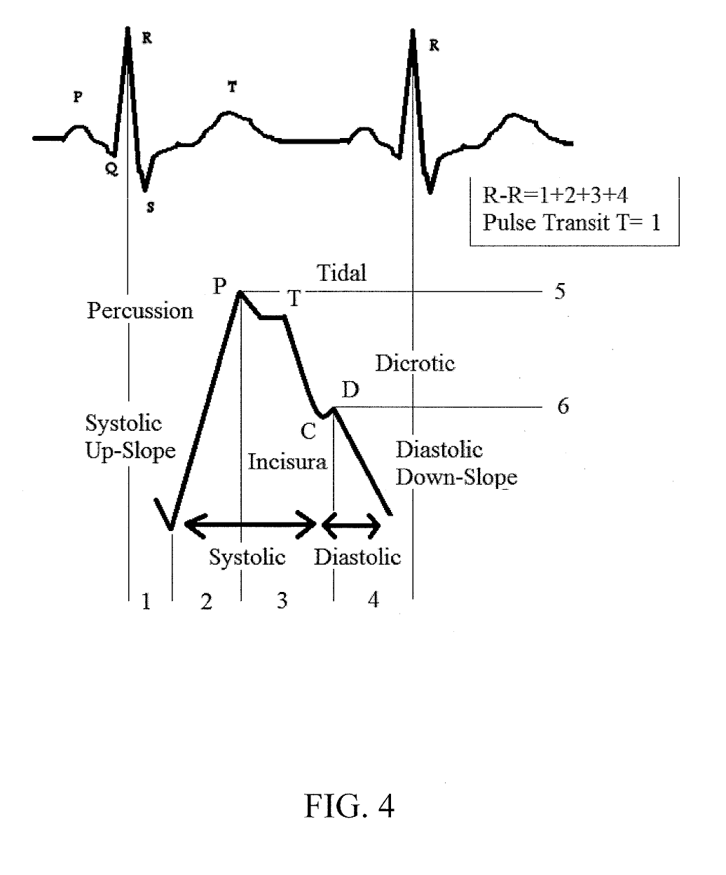

After acquiring the PPG and ECG signals, the preecampsia recognizer (e.g., RBF classifier) finds the corresponding pulses between both signal types. From these pulses the system aggregates a multitude of relative timing features from the signals. These include timing between pulses (T1+T2+T3+T4), timing from peak of the R-wave to the dicrotic notch (T1+T2+T3), timing from the dicrotic notch to the next R-wave (T4), timing from the R-wave to first dip in the PPG signal (of pulse) (T1). Additional time and frequency features are obtained by combining subset features and applying mathematical functions (derivative, log, ratios, FFT, etc.). For example, as illustrated in FIG. 4, the heart rate (A) is derived from 1/(average time between R waves) or (1/average 1+2+3+4)), and the pulse transit time (B) is T1. These features are combined to create a high-dimensional feature vector that is then used in a linear or non-linear method to discern the patient types (e.g., preeclamptic patients without symptoms or clinical signs).

In one embodiment, augmentation index-like parameters are combined with pulse transit time parameters (ECG-PPG timing between ECG beat and PPG beat—how long it takes for blood to get to arm/finger) to determine whether a patient has preeclampsia, including determining whether a non-symptomatic patient (or a patient without any demonstrable clinical signs) has preeclampsia. ECG signals provide heart rate, heart rate variability, and similar parameters. Combined ECG and PPG provide PTT as described above. PTT is known to correlate with blood pressure. In certain related embodiments, PTT, in relation to heart rate variability, provides a ratio that is useful in determining a patient with preeclampsia (whether or not the patient demonstrates any symptoms or clinical signs of preeclampsia). The PPG can also be used for pulse waveshape analysis such as location of the reflective wave relative to the primary wave.

In another embodiment of the invention, the QRS peak from an ECG signal is a feature that is applied to the high-dimensional feature vector in accordance with the subject invention. The QRS peak is used for heart rate, heart rate variability, and PTT timing. An advantage of the subject system and method is that to determine preeclampsia in a patient, neither the P or T waves of the ECG signal are required. Moreover, the finer detail of the ECG signal is also extraneous. Obtaining the QRS peak is the easiest part to capture in an ECG signal.

According to certain embodiments of the invention, combinations of timing parameters related to the feature of pulse information are features applied to a high-dimensional feature vector. For example the dicrotic notch or Pre-Ejection Period (PEP), PTT, and QRS (of the ECG) are features that can be applied to a feature vector. Other features that can apply either alone or in various combinations to a feature vector include, but are not limited to:
Time between QRS to rising slope of PPG
Time between QRS peaks
Time between dicrotic notch of the PPG and QRS peak
Time between QRS peak and the dicrotic notch of the PPG
Time between the percussion wave peak of the PPG to the QRS between pulses
Time between the rising slope of the PPG to the QRS
The height of the dicrotic notch of the PPG
The height of the percussion wave peak of the PPG
The height of the systolic wave of the PPG
Ratios of the 3 heights above

For all of these timing parameters, the mean and variance of the values are determined, as well as the “beat to beat” variability (variability of the successive differences of the parameters in the time series), before application to a feature vector.

In a pulse-oximeter, the system uses two wavelengths of light and analyzes the relationships of the two signals during the various phases of the cycle to come up with the oxygen saturation. Calculating the correct saturation requires good quality signals. Because the subject system and methods are primarily focused upon timing and secondarily on the shape of the pulse (and not saturation), a single wavelength is all that is required from a pulse-oximeter and the quality does not need to be high. Since the quality of the signal can be poor, a “reflective” sensor can be used (one that senses reflected light, versus transmitted light). Reflective sensors provide lower quality data but are more convenient since they can be used in places other than extremeties (the transmitted light sensors must be used on “thin” parts of the body, like fingers, ears, noses, etc). Accordingly, one embodiment of the invention comprises at least one optical transducer, wherein the optical transducer comprises reflective sensors.

Another embodiment of the sensor system is its ability to calculate arterial stiffness and blood pressure. These features may be used in conjunction with the preeclampsia detection system or separately.

Using the American College of Obstetricians and Gynecologists (ACOG) definition of severe preeclampsia, the system can distinguish severe preeclampsia from mild or other forms of hypertension. Severe preeclamptics require the most aggressive efforts to prevent poor outcomes or death for both the mother and fetus.

In an embodiment, particularly for high risk patients, the subject system can monitor the subject regularly (e.g. daily or weekly) or continuously and detect changes in the vascular or preterm labor status of the patient. Particularly in patients already determined likely to become preeclamptic, the system can monitor for impending symptoms or severity that would require a clinical (sometimes rapid) response. Trends in the data could be utilized to detect changes that required care such as the administration of supplements in developing nations or experimental therapies in the US. The intelligence system could be programmed with recommendations based on medical standards or previous or ongoing studies.

The system may also include methods for providing advice to the patient or clinician based on the output of the system. Methods such as fuzzy logic or rule-based systems provide the advice based on information gathered from the patient, information from clinicians, and information from the literature or standards. This information is combined by the system to provide the most relevant advice on treating the patient or preparing the patient for treatment.

The systems and methods of the invention can be used in: clinics, doctors' offices and emergency departments as a preeclampsia screening tool, in hospitals to confirm or rule-out preeclampsia in atypical presentations, and in developing nations where complications from preeclampsia are a leading cause of death, and patient transportation to an appropriate care facility poses a significant challenge. The prediction function would be invaluable in prenatal clinics for appropriate care plan development, particularly should the device predict future severe, early-onset preeclampsia in which preparation for delivery at a tertiary care center can be made. Finally, the potential for use of this device in ongoing research into prevention strategies cannot be over-stated. The ability to select only those patients destined to develop preeclampsia for clinical studies of supplements and interventions will increase the feasibility of such studies and reduce the cost of research.

### EXAMPLE 1

The following study was conducted to validate the ability of the system and method of the invention to identify preeclampsia in a patient. After written, informed consent, 66 women admitted to Labor & Delivery were studied with the distribution shown in the table below.

| Diagnosis | Average GA | N |
|---|---|---|
| Control | 36.2 | 27 |
| Gestational Hypertension | 38.3 | 4 |
| Chronic Hypertension | 33.9 | 9 |
| Chronic Hypertension with | 31.4 | 7 |
| Super-Imposed PreEclampsia | | |
| PreEclampsia | 33.1 | 19 |

Continuous ECG recording from the maternal chest and pulse oximetry waveforms (photoplethysmography, PPG) from the middle finger were obtained for 30-minutes with the patient at rest. Various timing features were obtained from each data set relative to the PPG and ECG signals. These features were then used as input into a Radial Basis Function Network (RBF, see FIG. 3) with a linear output to discriminate/detect preeclamptics versus controls. The RBF was trained with 1000 different trials utilizing different mixtures of training and cross validation data. The sensitivity of the system was 0.86, the PPV was 0.75, and the area under the curve (AUC) of the receiver operating characteristic (ROC) curve was 0.8. The combination of sensitivity and PPV is superior to any other research reported to date (excluding invasive, chemical, or biomarker methods) and has been achieved using a simple, inexpensive pulse-oximeter and ECG lead.

Simultaneously, antenatal data in the high risk OB clinic was collected. Inclusion criteria consist of women prior to 25 weeks gestation with multi-fetal gestation, chronic hypertension, pre-gestational diabetes, or history of preeclampsia in a prior pregnancy. After written informed consent, subjects underwent the same protocol as above for 30-minutes at each prenatal visit and again when they presented for delivery, if possible. Data was stored for subsequent analysis in light of delivery outcome. To date 26 women have enrolled. Of those, 11 have delivered: 7 with preeclampsia and 4 without. Using the term patients (control and preeclamptics described above) to train the RBF predictive model, 82% of subjects were correctly predicted at least 10 weeks before the onset of symptoms (or delivery). The RBF was trained with 1000 different trials utilizing different mixtures of training and cross validation data. The sensitivity of the system was 0.86, the PPV was 0.75, and the area under the curve (AUC) of the receiver operating characteristic (ROC) curve was 0.8. The combination of sensitivity and PPV is superior to any other research reported to date (excluding invasive, chemical, or biomarker methods) and has been achieved using a simple, inexpensive sensor device comprising a pulse-oximeter and ECG lead.

We claim:

1. A preeclampsia detection system comprising: two or more electrodes, one or more optical transducers, and a processor configured to run a preeclampsia recognizer, wherein the electrodes and transducer(s) are designed to non-invasively capture data regarding preeclampsia.

2. The system of claim 1, wherein the sensors are co-located in a single sensor device.

3. The system of claim 1, wherein the sensors are located in separate sensor devices.

4. The system of claim 1, wherein the one or more optical transducers are located in a pulse oximeter.

5. The system of claim 1, wherein the preeclampsia recognizer provides recommendations based on the data captured by the sensor device.

6. The system of claim 5, wherein the recommendations relate to the treatment of preeclampsia or methods of protecting the lives of the mother or fetus.

7. The system of claim 6, wherein the processor is also configured to run signal processing algorithms that extract features to model the onset of preeclampsia.

8. The system of claim 7, wherein the features include one or more of: heart rate, pulse transit time, augmentation indices, variability of heart rate, variability of pulse transit time, variability of augmentation indices, and combinations or ratios of the aforementioned features.

9. The system of claim 7 wherein one of the signal processing algorithms of the processor includes determining the onset of preeclampsia in a patient that has neither symptoms nor clinical signs of preeclampsia.

10. The system of claim 7, wherein the processor differentiates between mild and severe preeclampsia.

11. The system of claim 7, wherein one of the signal processing algorithms of the processor includes performing any one or more of: uterine activity monitoring, fetal heart rate monitoring, fetal ECG extraction, and preterm labor detection.

12. A sensor device comprising two or more electrodes, and one or more optical transducers.

13. The sensor device of claim 12, wherein the sensor device is portable and/or wearable.

14. The sensor device of claim 12, wherein the sensor device is modular and provided in the form of a wrist strap or arm band.

15. The sensor device of claim 12, further comprising a processor configured to run a preeclampsia recognizer, wherein the preeclampsia recognizer comprises a processor for running signal processing algorithms that extract features to model the onset of preeclampsia.

18. A method for detecting and/or predicting preeclampsia in a patient comprising the steps of:
(a) providing a preeclampsia detection system of claim 1 to a patient;
(b) analyzing information provided by the preeclampsia detection system; and
(c) identifying if the patient is preeclamptic.
